(19) United States
(12) Patent Application Publication
(10) Pub. No.: US 2022/0033344 A1
(43) Pub. Date: Feb. 3, 2022

(54) METHOD FOR PREPARING (2S,3S)-3-AMINO-BICYCLO[2.2.2]OCTANE-2-CARBOXYLATE

(71) Applicant: GUANGDONG RAFFLES PHARMATECH CO., LTD, Huizhou, Guangdong (CN)

(72) Inventors: Zhangtao ZHOU, Huizhou, Guangdong (CN); Zhining HUANG, Huizhou, Guangdong (CN); Weiping YE, Huizhou, Guangdong (CN); Andrew Toroa PHILLIS, Huizhou, Guangdong (CN)

(21) Appl. No.: 17/262,079

(22) PCT Filed: Jun. 30, 2020
(86) PCT No.: PCT/CN2020/099360

§ 371 Date: Jan. 21, 2021

(30) Foreign Application Priority Data
Dec. 30, 2019 (CN) 201911403717.X

(51) Int. Cl. C07C 227/18; C07C 227/32

(52) U.S. Cl. CPC C07C 227/18; C07C 2602/44; C07B 2200/07; C07C 227/32

(57) ABSTRACT

A method for preparing (2S,3S)-3-amino-bicyclo[2.2.2]octane-2-carboxylate is in the field of pharmaceutical intermediate synthesis. The method uses 3-carbonyl-bicyclo[2.2.2]octane-2-carboxylate as the starting material and performs reductive amination, alkalinity configuration flip, and hydrogenation to remove the protecting group in sequence to obtain the target product. This synthesis method of (2S,3S)-3-amino-bicyclo[2.2.2]octane-2-carboxylate is characterized by a novel route, mild reaction conditions and low cost, with a yield of more than 65%.

The present application claims the priority of the prior application No. 201911403717.X submitted to China National Intellectual Property Administration on Dec. 30, 2019, which is entitled “Method for preparing (2S,3S)-3-amino-bicyclo[2.2.2]octane-2-carboxylate”. The entire content of the prior application is incorporated herein by reference.

### FIELD OF THE INVENTION

The present disclosure belongs to the field of organic chemical synthesis of pharmaceutical intermediates, and specifically relates to a new method for the synthesis and industrialization of (2S,3S)-3-amino-bicyclo[2.2.2]octane-2-carboxylate using an in-house designed and innovative intermediate.

### BACKGROUND OF THE INVENTION

(2S,3S)-3-amino-bicyclo[2.2.2]octane-2-carboxylate belongs to a class of chiral small molecules that are difficult to synthesize. This chiral fragment is widely used in the manufacture of an influenza virus RNA polymerase inhibitors, the most representative of which is Pimodivir developed by Vertex, which has entered the clinical phase III study. The novel target of this class of drugs is a milestone in addressing influenza virus drug resistance.

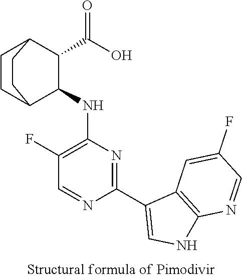 [SMILES: O=C(O)[C@H]1C2CCC(CC2)[C@@H]1Nc1nc(-c2c[nH]c3ncc(F)cc23)ncc1F]

Three main routes have been reported for the synthesis of this structural fragment of (2S,3S)-3-amino-bicyclo[2.2.2]octane-2-carboxylate as follows.

Route I is shown as the following chart “Route I reported in a patent”. This route starts with cyclohexadiene, undergoes Diels-Alder reaction with maleic anhydride, further selective alcoholysis in the presence of quinidine to obtain cis-carboxylic acid esters, flips the ester group conformation under strong base conditions, and finally performs Curtius rearrangement with diphenyl azide phosphate, and finally obtain the target product by removal of benzoxy carbonyl. Although the starting material is relatively cheap, the main shortcomings of this route include:

(1) requires the use of expensive chiral organic base quinidine for desymmetrizing alcoholysis, resulting in high costs;

(2) requires the use of explosive azide compounds, which is a safety hazard and not conducive to scaling up production;

(3) the total yield is less than 20%.

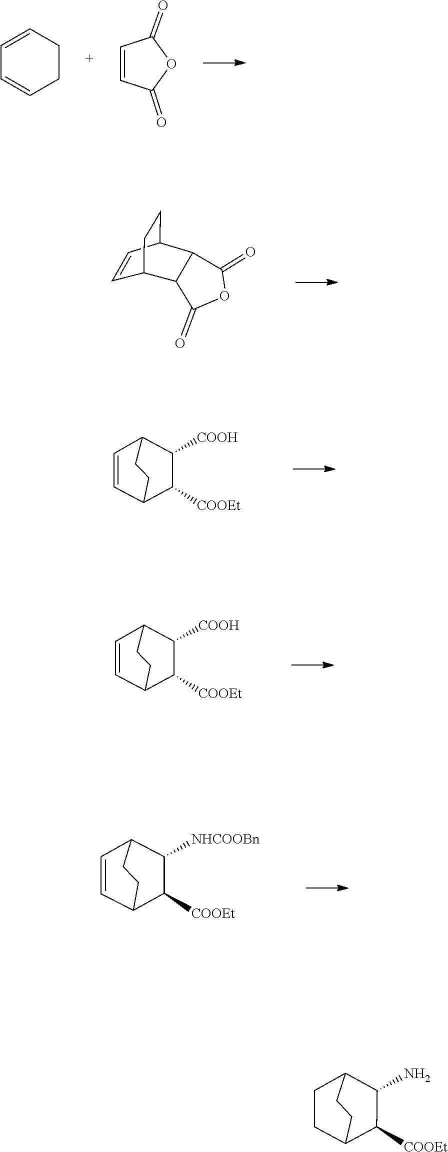

### Route I Reported in a Patent

Route II is shown as the following chart “Route II reported in a patent”. This route takes cyclohexadiene as the starting material, undergoes the Diels-Alder reaction with ethyl propargylate, and after further hydrogenation for selective reduction of the double bond, undergoes Michael addition reaction with chiral amine anion at low temperature, and finally removes the two protecting groups to obtain the target compound.

This route also has many shortcomings. Firstly, the starting materials are expensive and costly; secondly, special metal catalysts are used in the selective reduction of double bonds and harsh conditions such as ultra-low temperatures are used in the subsequent addition reactions, which are not conducive to scale-up production; the overall production cost is also high.

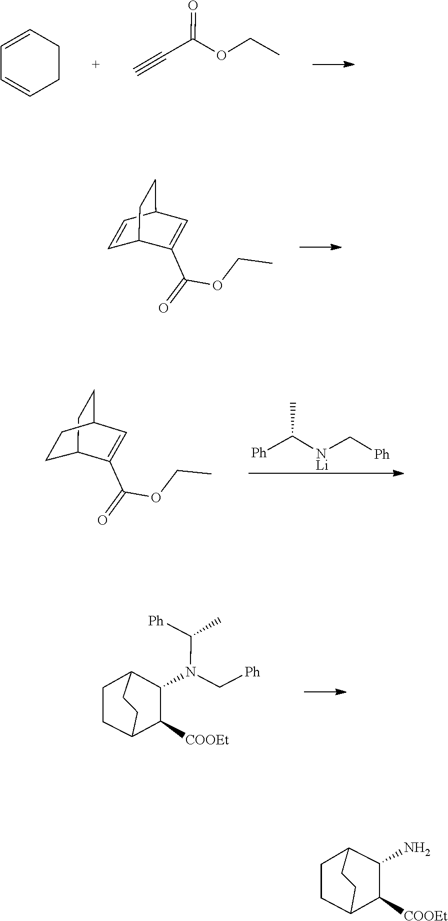

### Route II Reported in a Patent

Route III is shown as the following chart “Route III reported in a patent”. This route starts with ethyl glyoxylate, which undergoes the Henry reaction with nitromethane under alkaline conditions to obtain ethyl nitroacrylate by elimination reaction, which is further hydrogenated with cyclohexadiene by the Diels-Alder reaction catalyzed by chiral auxiliaries to obtain the target product.

Although this route is short, the raw materials are expensive and chemically unstable, while the use of nitromethane and nitro-containing intermediates poses a greater safety risk to the production.

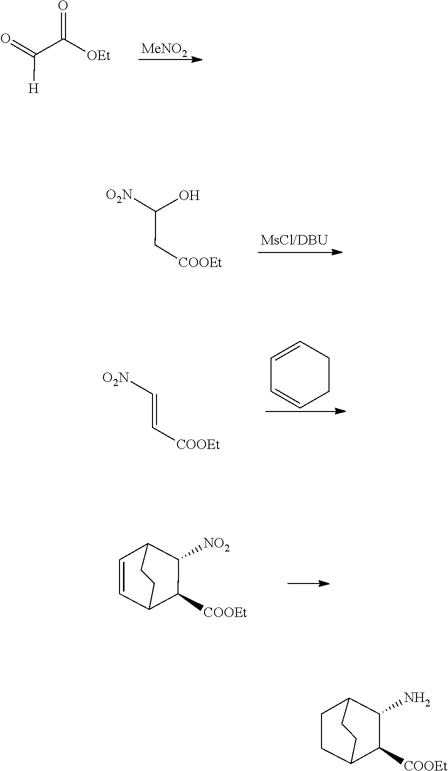

### Route III Reported in a Patent

In summary, the reported synthetic routes for (2S,3S)-3-amino-bicyclo[2.2.2]octane-2-carboxylate are characterized by high production risks and high costs, which make it difficult to meet the demand for this class of pharmaceutical intermediates in the pharmaceutical industry.

### SUMMARY OF THE INVENTION

To address the shortcomings of the prior art and to solve the problems of high preparation cost, low material safety and difficulty in production scale-up of (2S,3S)-3-amino-bicyclo[2.2.2]octane-2-carboxylate, the present invention provides a method for the preparation of (2S,3S)-3-amino-bicyclo[2.2.2]octane-2-carboxylate through two independently designed and innovative intermediates. The method utilizes a chiral reductive amination strategy and achieves excellent results.

The present disclosure provides a method for preparing (2S,3S)-3-amino-bicyclo[2.2.2]octane-2-carboxylate, the method comprises: using 3-carbonyl-bicyclo[2.2.2]octane-2-carboxylate as raw material, carrying out reductive amination, flip of ester group conformation and removal of protecting group to obtain the (2S,3S)-3-amino-bicyclo[2.2.2]octane-2-carboxylate, and reaction process is shown below.

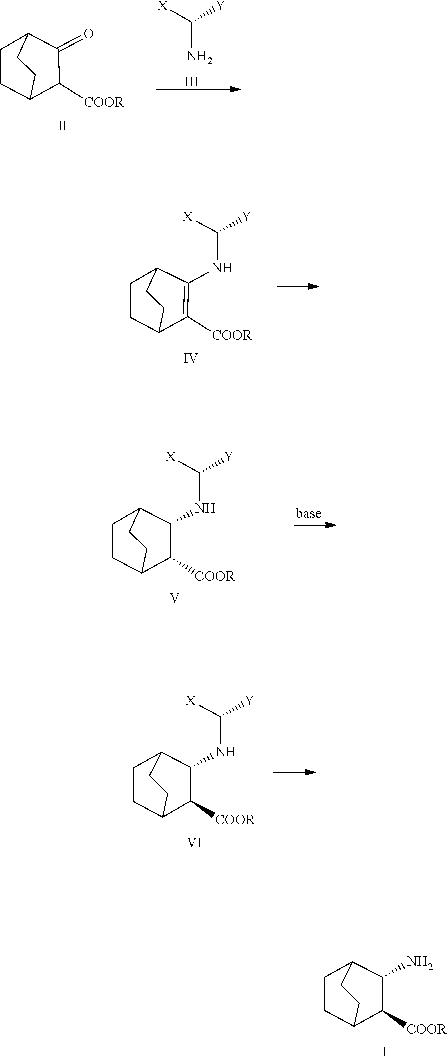

wherein, X, Y and R are all organic substituents.

According to an embodiment of the present invention, the method comprises:

S1, reacting 3-carbonyl-bicyclo[2.2.2]octane-2-carboxylate with chiral amines in presence of acid to give 3-amido-bicyclo[2.2.2]octene-2-carboxylate;

S2, carrying out reduction with a reducing agent or metal-catalyzed hydrogenation of the 3-amido-bicyclo[2.2.2]octene-2-carboxylate to give (2R,3S)-3-amido-bicyclo[2.2.2]octane-2-carboxylate;

S3, under strong base conditions, carrying out ester configuration flip of the (2R,3S)-3-amido-bicyclo[2.2.2]octane-2-carboxylate to give (2S,3S)-3-amido-bicyclo[2.2.2]octane-2-carboxylate;

S4, carrying out hydrogenation of the (2S,3S)-3-amido-bicyclo[2.2.2]octane-2-carboxylate to remove the protecting group to give the (2S,3S)-3-amino-bicyclo[2.2.2]octane-2-carboxylate.

According to an embodiment of the present invention, in the above route, the R is methyl, ethyl, propyl, butyl, phenyl or benzyl; the X is methyl, ethyl, phenyl or 1-naphthyl; the Y is methyl, ethyl, phenyl or 1-naphthyl; and the X and the Y are not identical, the X group is larger than the Y group. By “the X group is larger than the Y group”, it means that mass of the X group is greater than mass of the Y group, or relative molecular weight of the X group is greater than relative molecular weight of the Y group. In the above selection range of X and Y groups, the relative molecular weight of the X group is larger than that of the Y group, which makes the steric effect of the X group larger than that of the Y group, and the chiral amine (compound III) is of S-configuration, which is ultimately favorable to obtain a chiral S-configuration product.

According to an embodiment of the present invention, the X is preferably phenyl, the Y is preferably methyl and the R is preferably ethyl considering the availability of the materials.

According to an embodiment of the present invention, in the S1, the metal catalyst for hydrogenation reduction comprises one of platinum carbon, platinum dioxide and ruthenium metal catalyst; the reduction reagent comprises one of sodium borohydride, sodium triacetoxy borohydride, sodium cyanoborohydride and sodium trifluoroacetoxy borohydride.

According to an embodiment of the present invention, in the S2, the metal-catalyzed hydrogenation is carried out using a metal catalyst, the metal catalyst comprising at least one selected from the group consisting of platinum carbon, platinum dioxide and ruthenium metal catalyst; the reducing agent comprising at least one selected from the group consisting of sodium borohydride, sodium triacetoxy borohydride, sodium cyanoborohydride and sodium trifluoroacetoxy borohydride; preferably, to further improve the selectivity of the reaction, the metal catalyst is platinum dioxide; the reducing agent is sodium triacetoxy borohydride.

According to an embodiment of the present invention, in the S3, the strong base comprises at least one selected from the group consisting of sodium tert-butoxide, sodium tert-amylate, lithium diisopropylamide, lithium bis(trimethylsilyl)amide, sodium bis(trimethylsilyl)amide and potassium bis(trimethylsilyl)amide; preferably, considering price and availability of materials, the strong base is sodium tert-butoxide.

According to an embodiment of the present invention, in the S1, the acid is organic acid or inorganic acid; preferably, the acid is strong organic acid; further preferably, the acid comprises p-toluenesulfonic acid or trifluoroacetic acid.

The present disclosure provides a compound shown in formula V below, wherein, the R is methyl, ethyl, propyl, butyl, phenyl or benzyl, preferably ethyl; the X is methyl, ethyl, phenyl or 1-naphthyl, and the Y is methyl, ethyl, phenyl or 1-naphthyl, and the X and the Y are not identical and the X is larger than the Y, preferably, the X is phenyl, the Y is methyl,

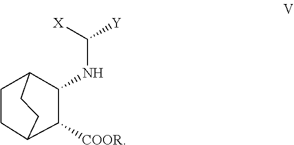

The present disclosure provides use of the compound V for preparation of (2S,3S)-3-amino-bicyclo[2.2.2]octane-2-carboxylate.

The present disclosure provides a compound shown in formula VI below, wherein, the R is methyl, ethyl, propyl, butyl, phenyl or benzyl, preferably ethyl; the X is methyl, ethyl, phenyl or 1-naphthyl, and the Y is methyl, ethyl, phenyl or 1-naphthyl, and the X and the Y are not identical and the X is larger than the Y, preferably, the X is phenyl, the Y is methyl,

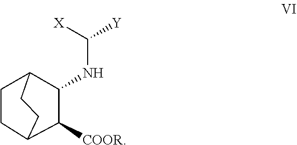

The present disclosure provides use of the compound VI for preparation of (2S,3S)-3-amino-bicyclo[2.2.2]octane-2-carboxylate.

Compared with the prior art, the present invention has the following advantages.

1. The synthetic route of the present invention is a novel route for the preparing of (2S,3S)-3-amino-bicyclo[2.2.2]octane-2-carboxylate. Two novel intermediates, namely, compound V and compound VI are obtained through the synthetic route of the present invention. An overall yield of more than 65% is achieved by the synthetic route of the present invention. The synthetic route of the present invention is also characterized by the short route with relatively mild reaction conditions.

2. High chiral purity intermediates can be obtained by the synthetic route provided by the present invention. The chiral purity of the target products can be increased to more than 99.5% after simple crystallization and purification.

3. Easily available raw materials are employed in the processes. The synthetic route of the present invention is low cost, requires no special operating process, employs simple equipments, and is suitable for large-scale industrial production.

### DETAILED DESCRIPTION OF THE INVENTION

The following is a detailed description of preferred embodiments of the present invention to make the advantages and features of the present invention more easily understood by those skilled in the art, so that the scope of protection of the present invention can be more clearly defined.

### Example 1

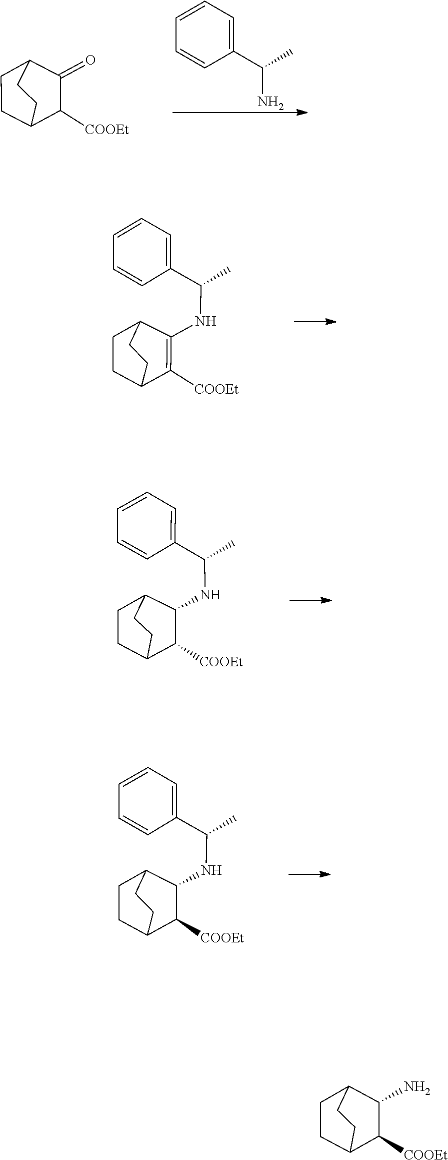

### 1. Synthesis of ethyl (S)-3-(1-phenylethylamino)-bicyclo[2.2.2]octene-2-carboxylate

To the reactor was added 1000 L toluene, 100.0 kg ethyl 3-carbonyl-bicyclo[2.2.2]octane-2-carboxylate, 12 kg p-toluenesulfonic acid, 80.0 kg S-1-phenylethylamine, and the reaction was refluxed under nitrogen protection for 12 h to obtain the enamine intermediate ethyl (S)-3-(1-phenylethylamino)-bicyclo[2.2.2]octene-2-carboxylate, which was used in the next reaction step.

### 2. Synthesis of ethyl (2R,3S)-3-((S)-1-phenylethylamino)-bicyclo[2.2.2]octane-2-carboxylate

The above enamine intermediate (S)ethyl-3-(1-phenylethylamino)-bicyclo[2.2.2]octene-2-carboxylate solution was desolvated, then 1500 L of tetrahydrofuran and 500 L of acetic acid was added, then 106.2 kg of sodium triacetoxyborohydride was added after cooling. Bring to room temperature and reacted for 3 h. 3N sodium hydroxide solution was added dropwise to adjust to alkaline, extracting with ethyl acetate (800 Lx 2), the combined organic phases were washed with saturated salt water and concentrated to give 115 g of ethyl (2R,3S)-3-((S)-1-phenylethylamino)-bicyclo[2.2.2]octane-2-carboxylate (corresponding to compound IV in the synthetic route of the present invention) as a pale yellow oil, in yield 85%. ¹HNMR (400 MHz, CDCl₃) δ7.22-7.32 (m, 5H), δ4.18 (q, 2H), δ3.65 (q, 1H), δ2.81-2.89 (m, 2H), δ1.83 (m, 2H), δ1.27-1.56 (m, 11H), δ1.25 (t, 3H); ESI-MS: m/z 302.34 [M+1]

### 3. Synthesis of ethyl (2S,3S)-3-((S)-1-phenylethylamino)-bicyclo[2.2.2]octane-2-carboxylate

To the reactor was added 500 L tetrahydrofuran, 500 L tert-butanol, 64 kg sodium tert-butoxide, and cooled to 0-10° C. under nitrogen protection. Add tetrahydrofuran solution of ethyl (2R,3S)-3-((S)-1-phenylethylamino)-bicyclo[2.2.2]octane-2-carboxylate (100 kg dissolved in 100 L tetrahydrofuran) dropwise. After dropwise addition, the reaction was held for 2 h. The reaction solution was transferred to 500 L saturated ammonium chloride solution for quenching. After extraction with ethyl acetate (800 L×2), the combined organic phases were washed with saturated brine and concentrated to give 90.0 kg of ethyl (2S,3S)-3-((S)-1-phenylethylamino)-bicyclo[2.2.2]octane-2-carboxylate (corresponding to compound V in the synthetic route of the present invention) as pale yellow oily form with 90.0% yield and diastereomeric purity 97.4%.

¹HNMR (400 MHz, CDCl₃) δ7.21-7.31 (m, 5H), δ4.13 (q, 2H), δ3.79 (q, 1H), δ3.12 (d, 1H), δ2.22 (d, 1H), δ1.93 (d, 1H), δ1.42-1.76 (m, 8H), δ1.23-1.34 (m, 8H); ESI-MS: m/z 302.34 [M+1]

### 4. Synthesis of ethyl (2S,3S)-3-amino-bicyclo[2.2.2]octane-2-carboxylate

500 L of ethanol, 8.00 kg of ethyl (2S,3S)-3-((S)-1-phenylethylamino)-bicyclo[2.2.2]octane-2-carboxylate, and 8 kg of 10% palladium carbon were added to a 1000 L stainless steel autoclave. The reactor was evacuated and full filled with nitrogen, then exchanged to hydrogen and pressurized to 0.6 MPa. The reaction mixture was heated to 50° C. and kept for 12 hours. The palladium carbon was removed by filtration and the filtrate was concentrated to give 50 kg of ethyl (2S,3S)-3-amino-bicyclo[2.2.2]octane-2-carboxylate (corresponding to compound I in the synthetic route of the present invention) as a pale yellow oil in 97.0% yield and 97.5% chiral purity.

¹HNMR (400 MHz, CDCl₃) δ4.18 (q, 2H), δ3.37-3.38 (m, 1H), δ2.13-2.17 (m, 3H), δ1.98-2.00 (m, 1H), δ1.78-1.83 (m, 1H), δ1.36-1.67 (m, 9H), δ1.27 (t, 3H); ESI-MS: m/z 198.26 [M+1]

### Example 2

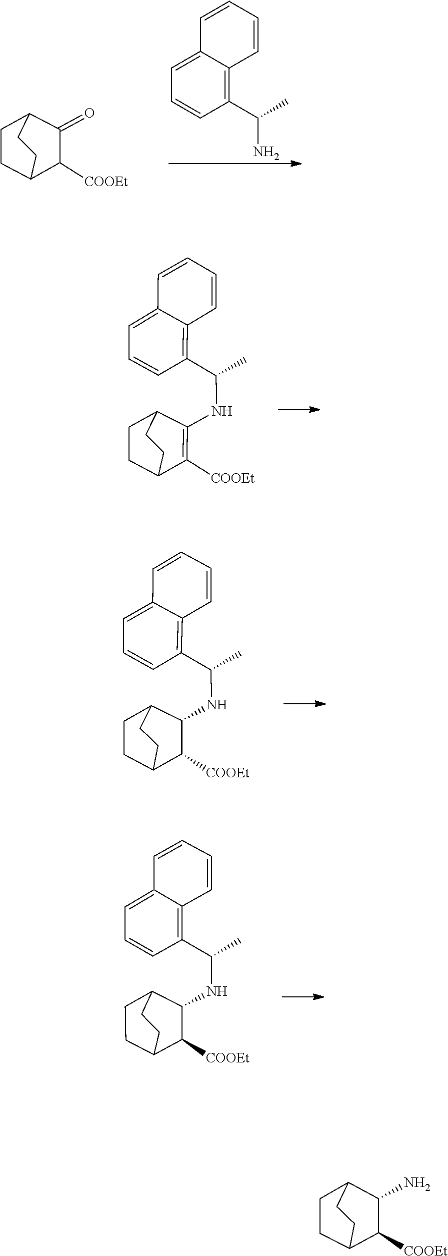 [SMILES: CCOC(=O)C1C(=O)C2CCC1CC2 ; C[C@H](N)c1cccc2ccccc12 ; CCOC(=O)C1=C(N[C@@H](C)c2cccc3ccccc23)C2CCC1CC2 ; CCOC(=O)[C@@H]1C2CCC(CC2)[C@@H]1N[C@@H](C)c1cccc2ccccc12 ; CCOC(=O)[C@H]1C2CCC(CC2)[C@@H]1N[C@@H](C)c1cccc2ccccc12 ; CCOC(=O)[C@H]1C2CCC(CC2)[C@@H]1N]

### 1. Synthesis of ethyl (S)-3-(1-naphthylethylamino)-bicyclo[2.2.2]octene-2-carboxylate

1000 L of toluene, 100.0 kg of ethyl 3-carbonyl-bicyclo[2.2.2]octane-2-carboxylate, 10.0 kg of trifluoroacetic acid, 113 kg of S-1-naphthyl ethylamine were added to the reactor and reacted under nitrogen protection at reflux for 12 hours. Cooled to room temperature, washed with 300 L saturated sodium bicarbonate solution, the organic layer was concentrated to 200 L, 500 L n-heptane was added and stirred for 3 h at room temperature, filtered, washed with a small amount of n-heptane and dried under vacuum to give 133.6 kg ethyl (S)-3-(1-naphthylethylamino)-bicyclo[2.2.2]octene-2-carboxylate as a white solid in 75.3% yield.

### 2. Synthesis of ethyl (2R,3S)-3-((S)-1-naphthylethylamino)-bicyclo[2.2.2]octane-2-carboxylate

300 L of ethanol, 200 L of ethyl acetate, 100.0 kg of ethyl (S)-3-(1-naphthylethylamino)-bicyclo[2.2.2]octene-2-carboxylate, 10.0 kg of 5% platinum carbon to 1000 L were added to stainless steel autoclave, degassing with nitrogen and then ventilating with hydrogen to 1 MPa, then the mixture was raised to 35° C. and kept for 10 hours. Filtered to remove the platinum carbon and the filtrate was concentrated to obtain 100.0 kg ethyl (2R,3S)-3-((S)-1-naphthylethylamino)-bicyclo[2.2.2]octane-2-carboxylate as an off-white solid in 99.4% yield.

### 3. Synthesis of ethyl (2S,3S)-3-((S)-1-naphthylethylamino)-bicyclo[2.2.2]octane-2-carboxylate

500 L tetrahydrofuran, 500 L tert-butanol, 50.0 kg sodium tert-butoxide were added to the reactor, cooled down to 0-10° C. under nitrogen protection, and added dropwise a tetrahydrofuran solution of ethyl (2R,3S)-3-((S)-1-naphthylethylamino)-bicyclo[2.2.2]octane-2-carboxylate (100.0 kg dissolved in 100 L tetrahydrofuran). After dropwise addition, the reaction was held for 2 h. The reaction solution was quenched by pouring into 500 L saturated ammonium chloride solution, then extracted with ethyl acetate (800 L×2). The combined organic phases were washed with saturated brine and concentrated to give 92.0 kg of ethyl (2S,3S)-3-((S)-1-naphthylethylamino)-bicyclo[2.2.2]octane-2-carboxylate as a pale yellow solid in 92.0% yield and 98.0% diastereomeric purity.

### 4. Synthesis of ethyl (2S,3S)-3-amino-bicyclo[2.2.2]octane-2-carboxylate

To a 1000 L stainless steel hydrogenator was added 500 L of ethanol, 90.0 kg of ethyl (2S,3S)-3-((S)-1-naphthylethylamino)-bicyclo[2.2.2]octane-2-carboxylate, 9 kg of 10% palladium carbon. After degassing with nitrogen, then ventilating with hydrogen to 1 MPa and the reaction was carried out at 50° C. for 12 h. The palladium carbon was removed by filtration and the filtrate was concentrated to give 50.0 kg of ethyl (2S,3S)-3-amino-bicyclo[2.2.2]octane-2-carboxylate as a pale yellow oil in 99.0% yield and 98.1% chiral purity.

1. Method for preparing (2S,3S)-3-amino-bicyclo[2.2.2]octane-2-carboxylate, wherein, the method comprises: using 3-carbonyl-bicyclo[2.2.2]octane-2-carboxylate as raw material, carrying out reductive amination, flip of ester group conformation and removal of protecting group to obtain the (2S,3S)-3-amino-bicyclo[2.2.2]octane-2-carboxylate, and reaction process is shown below, 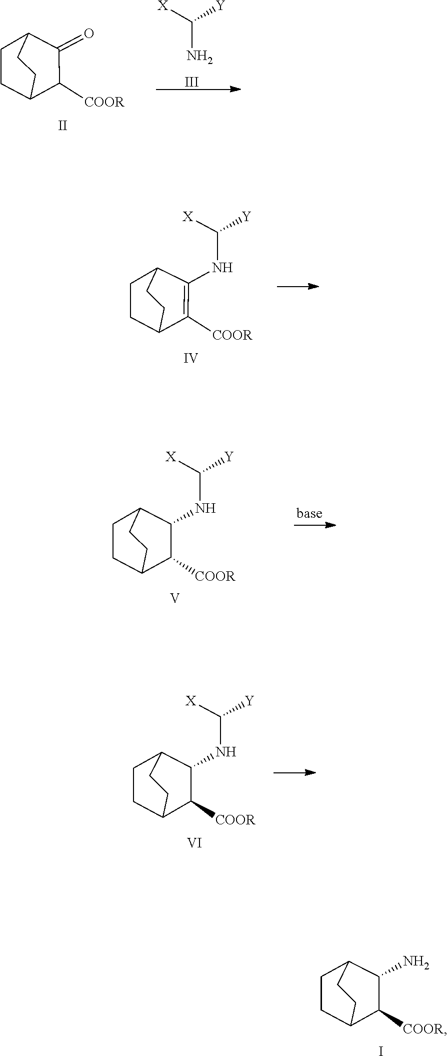
wherein, X, Y and R are all organic substituents.

2. The method according to claim 1, wherein, the method comprises:
S1, reacting 3-carbonyl-bicyclo[2.2.2]octane-2-carboxylate with chiral amines in presence of acid to give 3-amino-bicyclo[2.2.2]octene-2-carboxylate;
S2, carrying out reduction with a reducing agent or metal-catalyzed hydrogenation of the 3-amino-bicyclo[2.2.2]octene-2-carboxylate to give (2R,3S)-3-amino-bicyclo[2.2.2]octane-2-carboxylate;
S3, under strong base conditions, carrying out ester configuration flip of the (2R,3S)-3-amino-bicyclo[2.2.2]octane-2-carboxylate to give (2S,3S)-3-amino-bicyclo[2.2.2]octane-2-carboxylate;
S4, carrying out hydrogenation of the (2S,3S)-3-amino-bicyclo[2.2.2]octane-2-carboxylate to remove the protecting group to give the (2S,3S)-3-amino-bicyclo[2.2.2]octane-2-carboxylate.

3. The method according to claim 1, wherein,
the R is methyl, ethyl, propyl, butyl, phenyl or benzyl, preferably ethyl;
the X is methyl, ethyl, phenyl or 1-naphthyl, the Y is methyl, ethyl, phenyl or 1-naphthyl, and the X and the Y are not identical, the X group is larger than the Y group;
preferably, the X is phenyl, the Y is methyl.

4. The method according to claim 2, wherein, in the S2, the metal-catalyzed hydrogenation is carried out using a metal catalyst, the metal catalyst comprising at least one selected from the group consisting of platinum carbon, platinum dioxide and ruthenium metal catalyst; the reducing agent comprising at least one selected from the group consisting of sodium borohydride, sodium triacetoxy borohydride, sodium cyanoborohydride and sodium trifluoroacetoxy borohydride; preferably, the metal catalyst is platinum dioxide; the reducing agent is sodium triacetoxy borohydride.

5. The method according to claim 2, wherein, in the S3, the strong base comprises at least one selected from the group consisting of sodium tert-butoxide, sodium tert-amylate, lithium diisopropylamide, lithium bis(trimethylsilyl)amide, sodium bis(trimethylsilyl)amide and potassium bis(trimethylsilyl)amide; preferably, the strong base is sodium tert-butoxide.

6. The method according to claim 2, wherein, in the S1, the acid is organic acid or inorganic acid; preferably, the acid is strong organic acid; further preferably, the acid comprises p-toluenesulfonic acid or trifluoroacetic acid.

7. A compound of formula V, wherein, the R is methyl, ethyl, propyl, butyl, phenyl or benzyl, preferably ethyl; the X is methyl, ethyl, phenyl or 1-naphthyl, and the Y is methyl, ethyl, phenyl or 1-naphthyl, and the X and the Y are not identical and the X is larger than the Y, preferably, the X is phenyl, the Y is methyl,

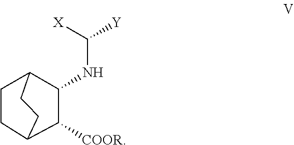

8. Use of the compound V according to claim 7 for preparation of (2S,3S)-3-amino-bicyclo[2.2.2]octane-2-carboxylate.

9. A compound of formula VI, wherein, the R is methyl, ethyl, propyl, butyl, phenyl or benzyl, preferably ethyl; the X is methyl, ethyl, phenyl or 1-naphthyl, and the Y is methyl, ethyl, phenyl or 1-naphthyl, and the X and the Y are not identical and the X is larger than the Y,
preferably, the X is phenyl, the Y is methyl,

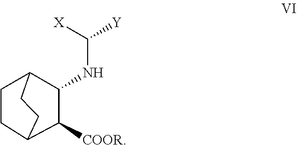

10. Use of the compound VI according to claim 9 for preparation of (2S,3S)-3-amino-bicyclo[2.2.2]octane-2-carboxylate.
